(19) United States
(12) Patent Application Publication
(10) Pub. No.: US 2006/0014145 A1
(43) Pub. Date: Jan. 19, 2006

(54) Methods and articles for high throughput analysis of biomolecular interactions

(76) Inventor: Brian Scott Miller, Palo Alto, CA (US)

Correspondence Address: H. Thomas Anderton;Genencor International, Inc., 925 Page Mill Road, Palo Alto, CA, 94304-1013, US

(21) Appl. No.: 10/508,199

(22) PCT Filed: Mar. 21, 2003
(86) PCT No.: PCT/US03/08774

§ 371 Date: Aug. 1, 2005

Related U.S. Application Data
(60) Provisional application No. 60/375,641, filed on Apr. 26, 2002.

(51) Int. Cl. C12Q 1/68; C12M 1/34

(52) U.S. Cl. 435/6; 435/287.2

(57) ABSTRACT

Methods, compositions articles of manufacture, and kits for characterizing biomolecular interactions are provided. The interactions may involve various combinations of proteins, lipids, carbohydrates and small molecules. For example, interactions between polynucleotide segments and polynucleotide binding protein(s) may be characterized.

### TECHNICAL FIELD

This invention relates to methods for the analysis of biomolecular interactions including those involving proteins, nucleic acids, and other cellular constituents.

### BACKGROUND OF THE INVENTION

Biomolecular interactions involving proteins, nucleic acids, lipids, carbohydrates, and small molecules lay at the heart of the biochemical processes underlying normal and abnormal physiology. Transcription factors are sequence-specific DNA binding proteins that positively regulate gene expression. Transcription factors bind promoter proximal elements and initiate the expression of target genes. They are modular proteins composed of distinct functional domains. Two domains are required for transcription factor function, i.e., a DNA-binding domain, and an activation domain. DNA-binding domains can be classified into a number of structural types including zinc-finger proteins, leucine zipper proteins, and homeodomain proteins. Transcription factors are but one example of polynucleotide binding proteins (“PBPs”) that interact with nucleic acid segment in a sequence-specific manner. Similar examples exist throughout phylogeny in plants, bacteria, yeast, fungi, etc.

Identifying the molecular participants in these and other types of biomolecular interactions is a necessary first step for developing a more detailed understanding of the mechanisms by which complex biochemical reactions proceed in normal and diseased states. Such an understanding is useful for identifying molecular targets which may be the subject of drug-discovery efforts. On one level, this type of interaction mapping has proceeded to identify groups of interacting proteins that may be involved in signal transduction, in combining to make molecular machines, that act together to provide structural definition to cells, or to carry out any of the other interactions needed to support life.

Currently, high throughput analysis of biological macromolecules typically centers on the analysis of transcription (mRNA levels, see TIBTECH March 1999 vol 17 pp 127-134), protein expression (proteomics, see Electrophoresis 2000, 21, 1071-1081) and protein-protein interaction (two-hybrid systems, see Nature, 2000, Vol 403, 623-627).

Diverse methods for developing protein-protein interaction maps include chemical cross-linking in which multi-functional reagents are used to covalently link together two protein molecules that are in close physical proximity, fluorescence resonance energy transfer to measure molecular distances between neighboring proteins, and yeast two-hybrid systems to drive reporter gene expression when two proteins interact.

Each of these methods suffers from one or more shortcomings addressed by the methods of the present invention. For example, chemical cross-linking experiments usually require large amounts of purified protein. Similarly, fluorescence energy resonance transfer experiments usually require large amounts of purified proteins that are is derivatized with a fluorophore, or cloned and isolated genes into which fluorescent domains may be inserted. Yeast two hybrid systems also require construction of expression libraries and transduction of those libraries into host cells. As such, none of these techniques is amenable for practice in the absence of large amounts of purified proteins, prior identification of at least one of the proteins participating in a biomolecular interaction, or expression libraries encoding the proteins of interest. Similarly, each of these methods is deficient insofar as it is difficult or impossible to easily conduct many assays in parallel to speed the analysis.

Interactions between polynucleotides and proteins that recognize them (polynucleotide binding proteins or “PBPs”) also constitute an important class of biomolecular interactions. Proteins that bind to polynucleotides are involved in widespread areas of biological function. Polynucleotide binding proteins (PBPs) in many cases have critical roles in regulating genome content, replication, transcription, RNA processing, RNA stability, sensing of infection and DNA and RNA degradation. Polynucleotide binding proteins may be important in regulation of gene activity and many other critical cellular and developmental pathways especially considering their role in regulating gene expression in normal physiological states and in gene dysregulations that underlie neoplastic diseases.

Certain DNA binding proteins have been well-studied. For example, the global regulator AbrB from Bacillus subtilis is one bacterial DNA binding protein that has been well characterized (J. Mol. Biol. 2001, Jan. 19; 305(3): 429-39). A number of DNA binding proteins have also been examined in eukaryotes, where greater than 17% of proteins may be imported in the nucleus to interact with DNA (EMBO Reports 2000, vol. 1, no. 5, pp 411-415). Detailed knowledge of DNA-protein interactions may allow pharmaceutical control of gene expression at the genetic level.

Techniques for studying polynucleotide binding proteins have remained fairly primitive. See, e.g., Eur. J. Biochem. 1987, vol. 166, pp 351-355. These methods are slow and laborious. Available array methods for detecting proteins typically involve binding of the protein to a small region on a detection surface such as a capture substrate. These regions bind only a small amount of protein and do not provide sufficient bound protein to allow any significant amounts of structural information to be obtained.

These methods for identifying participants in protein-polynucleotide interactions therefore also suffer from many of the same shortcomings as those used for characterizing protein-protein interaction insofar as they may require large amounts of materials, a priori knowledge of the identity of one of the interacting members, construction of expression libraries or reporter constructs, and are not easily carried out in a multiplexed format. For example, Kauffman et al. in U.S. Pat. No. 6,100,035 (incorporated herein by reference) describe a method of identifying cis acting nucleic acid elements that involves contacting a nucleic acid binding factor preparation with a diverse population of nucleic acids on a solid support, and detecting which nucleic acids have been specifically bound by a nucleic acid binding factor. This method suffers from various difficulties addressed by the present invention including difficulties in obtaining adequate amounts of bound nucleic acid binding factors, thus precluding identification of nucleic acid binding factors that may be present in low concentration or amount.

Thus, there is a need in the art for methods of analyzing biomolecular interactions that may be carried out easily and rapidly and that are capable of identifying interacting members that may be present only as minor components in a mixture of similar components. The present invention addresses these and other shortcomings of the prior art by providing such methods, along with devices, compositions, kits, and articles of manufacture useful in the practice of such methods.

### SUMMARY OF THE INVENTION

Methods, compositions and articles for characterizing participants in a biomolecular interaction are provided. In one aspect, the invention provides a method of identifying biomolecular interactors comprising providing a mixture of capture substrates wherein each substrate comprises an identifier uniquely associated with a distinct biomolecule. The capture substrate mixture is contacted with a sample comprising at least one other biomolecule under conditions in which the biomolecules on the capture substrates may bind to one or more biomolecules in the sample. The capture substrates are sorted to collect substrates having the same identifier. At least one sample biomolecule bound to a capture substrate biomolecule is characterized.

In preferred embodiments, the capture substrate biomolecules and the sample biomolecules are selected from the group consisting of proteins, nucleic acids, lipids, carbohydrates, and small molecules. In an especially preferred embodiment, the capture substrate biomolecule is a nucleic acid and the sample biomolecule is a polynucleotide binding protein (“PBP”). In another preferred embodiment, the capture substrate biomolecule is a polynucleotide binding protein and the sample biomolecule is a nucleic acid. In yet another preferred embodiment, both the capture substrate biomolecule and the sample biomolecule are proteins.

In preferred embodiments, the methods may be practiced to identify the binding profile of a naturally occurring or manipulated polynucleotide binding protein against a plurality of predetermined capture substrate polynucleotide segments, to profile the different types of PBPs present in a particular sample (e.g., a unicellular organism, a cell type, or a tissue), and to provide structural information about those PBPs. The methods may be used to characterize any PBPs that bind to regions of genomic DNA of interest, including the entire genome of a given organism. The invention may also be used as an alternative to known methods for characterizing polynucleotide binding proteins and for characterizing protein-protein interactions. Kits comprising reagents useful for performing the methods of the invention are also provided.

### DETAILED DESCRIPTION OF THE PREFERRED EMBODIMENTS

### DEFINITIONS

In describing the present invention, the following terms will be employed, and are intended to be defined as indicated below.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein may be practiced in the practice or testing of the invention, the preferred methods and materials are described.

Terms such as “connected,” “attached,” “linked,” and “conjugated” are used interchangeably herein and encompass direct as well as indirect connection, attachment, linkage or conjugation unless the context clearly dictates otherwise.

The term “uniquely associated” means there is a one-to-one correspondence between two or more properties such that the two or more properties invariably are found together.

Where a range of values is recited, it is to be understood that each intervening integer value, and each fraction thereof, between the recited upper and lower limits of that range is also specifically disclosed, along with each subrange between such values. The upper and lower limits of any range may independently be included in or excluded from the range, and each range where either, neither or both limits are included is also encompassed within the invention. Where a value being discussed has inherent limits, for example where a component may be present at a concentration of from 0 to 100%, or where the pH of an aqueous solution may range from 1 to 14, those inherent limits are specifically disclosed. Where a value is explicitly recited, it is to be understood that values which are about the same quantity or amount as the recited value are also within the scope of the invention.

The terms “polynucleotide,” “polynucleotide segment,” “oligonucleotide,” “nucleic acid” and “nucleic acid molecule” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hnRNA, and mRNA, whether spliced or unspliced, any other type of polynucleotide which is an N— or C-glycoside of a purine or pyrimidine base, and other polymers containing nonnucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides may also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

Furthermore, modifications to nucleotidic units include rearranging, appending, substituting for or otherwise altering functional groups on the purine or pyrimidine base which form hydrogen bonds to a respective complementary pyrimidine or purine. The resultant modified nucleotidic unit optionally may form a base pair with other such modified nucleotidic units but not with A, T, C, G or U. Abasic sites may be incorporated which do not prevent the function of the polynucleotide. Some or all of the residues in the polynucleotide may optionally be modified in one or more ways.

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′-H and C6-oxy, respectively, of guanosine. Thus, for example, guanosine (2-amino-6-oxy-9-β-D-ribofuranosyl-purine) may be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al. (1993), supra, and Mantsch et al. (1993) Biochem. 14:5593-5601, or by the method described in U.S. Pat. No. 5,780,610 to Collins et al. Other nonnatural base pairs may be synthesized by the method described in Piccirilli et al. (1990) Nature 343:33-37 for the synthesis of 2,6-diaminopyrimidine and its complement (1-methylpyrazolo-[4,3]pyrimidine-5,7-(4H,6H)-dione. Other such modified nucleotidic units which form unique base pairs are known, such as those described in Leach et al. (1992) J. Am. Chem. Soc. 114:3675-3683 and Switzer et al., supra.

“Preferential binding” or “preferential hybridization” refers to the increased propensity of one member of a binding pair to bind to a second member of a binding pair as compared to other molecules present in the sample under the reaction conditions used.

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include polypeptides contain co- and/or post-translational modifications of the polypeptide, for example, glycosylations, acetylations, phosphorylations, and sulphations. In addition, protein fragments, analogs (including amino acids not encoded by the genetic code, e.g. homocysteine, ornithine, D-amino acids, and creatine), natural or artificial mutants or variants or combinations thereof, fusion proteins, derivatized residues (e.g. alkylation of amine groups, acetylations 15 or esterifications of carboxyl groups) and the like are included within the meaning of polypeptide.

As used herein, the term “binding pair” refers to first and second molecules that bind specifically to each other with greater affinity than to other components in the sample. The binding between the members of the binding pair is typically noncovalent. Exemplary binding pairs include a polynucleotide-binding protein and a polynucleotide comprising a binding site for that PBP, immunological binding pairs (e.g. any haptenic or antigenic compound in combination with a corresponding antibody or binding portion or fragment thereof, for example digoxigenin and anti-digoxigenin, fluorescein and anti-fluorescein, dinitrophenol and anti-dinitrophenol, bromodeoxyuridine and anti-bromodeoxyuridine, mouse immunoglobulin and goat anti-mouse immunoglobulin) and nonimmunological binding pairs (e.g., biotin-avidin, biotin-streptavidin, hormone [e.g., thyroxine and cortisol]-hormone binding protein, receptor-receptor agonist or antagonist (e.g., acetylcholine receptor-acetylcholine or an analog thereof) IgG-protein A, lectin-carbohydrate, enzyme-enzyme cofactor, enzyme-enzyme-inhibitor, and complementary polynucleotide pairs capable of forming nucleic acid duplexes) and the like. One or both member of the binding pair may be captured to additional molecules.

The terms “substrate” and “support” are used interchangeably and refer to a material having a rigid or semi-rigid surface having a propensity to assume a particular shape.

A “capture substrate” is a substrate linked to a biomolecule such as a protein, a nucleic-acid, a lipid, a carbohydrate, or a small molecule. For example, a capture substrate is “linked” or “conjugated” to, or chemically “associated” with, a polynucleotide when the capture substrate is coupled to, or physically associated with the polynucleotide. Thus, these terms intend that the capture substrate may either be directly linked to the polynucleotide or may be linked via a linker moiety, such as via a chemical linker. The terms indicate items that are physically linked by, for example, covalent chemical bonds, physical forces such van der Waals or hydrophobic interactions, encapsulation, embedding, or the like. For example, capture substrates comprising transponder capture substrates may be associated with biotin which may bind to the proteins avidin and streptavidin, which may be linked to a polynucleotide.

A “polynucleotide binding protein” or “PBP” refers to a protein that recognizes and binds to a polynucleotide in a sequence-specific manner.

An “identifier” refers to anything that may be practiced to distinguish capture substrates so that a mixed population of capture substrates comprising different identifiers may be sorted into collections of capture substrates having the same identifier. In the practice of the present invention, an identifier is uniquely associated with a distinct biomolecule, which of course permits the sorting to collect together substrates having the same identifier and by virtue of the identifier/biomolecule association, the same biomolecule. Optionally, the identity of a biomolecule associated with a given identifier may be known so that the identifier may be practiced to identify the biomolecule (i.e., identify the particular protein, particular polynucleotide segment, etc.) associated with the identifier.

“Multiplexing” refers to an assay or other analytical method in which multiple biomolecules may be assayed in parallel for their abilities to bind one another.

A “transponder” refers to a wireless device that picks up and automatically responds to an incoming signal. A transponder may be physically tiny (having dimensions on the order of microns or less). Transponders input (input) frequencies and output (transmitter) frequencies may be pre-assigned. A group of transponders used in the practice of the invention may have the same input frequency, but must generate output signals that are distinguishable from each other.

A “fluorophore” refers to a substance that absorbs electromagnetic radiation at a first wavelength, usually ultraviolet light, and releases the absorbed energy by emitting light having a longer wavelength. Absorption of electromagnetic energy by the fluorophore kicks an electron of an atom within the fluorophore from a lower energy state into an “excited” higher energy state; then the electron releases the energy in the form of light when it falls back to a lower energy state.

### Methods for Practicing the Preferred Embodiments

The invention described herein is useful for any assay to characterize the binding of one or more biomolecules present in a sample to another biomolecule. In preferred embodiments the biomolecules are selected from the group consisting of proteins, nucleic acids, lipids, carbohydrates, and small molecules. In one preferred embodiment the biomolecules comprise proteins.

In another preferred embodiment, the biomolecules comprise proteins and nucleic acids. While this embodiment is described with reference to an assay setup in which capture substrates comprise polynucleotide segments (i.e., nucleic acids) and the sample comprises proteins (i.e., polynucleotide binding proteins, or PBPs), one of ordinary skill will readily appreciate that the alternative setup in which the capture substrates comprise proteins and the sample comprises polynucleotide segments may also be practiced and falls within the scope of the instant invention.

In a preferred embodiment, a sample comprising at least one PBP is contacted with a mixture of capture substrates under conditions in which the polynucleotide binding protein(s) may bind to polynucleotide segments on the capture substrates. As described above, the capture substrates also comprise identifiers that are uniquely associated with a distinct polynucleotide segment. The contacting occurs in a liquid medium. Sorting is used to separate different capture substrates comprising different identifiers and to collect capture substrates having the same identifier. The sorting may be carried out manually (e.g., under circumstances in which the different identifiers may be distinguished by eye or through the use of a device that is manually operated) or the sorting may be automated through the use of, for example, flow sorting techniques. Automated sorting systems may include pneumatic or electroosmotic liquid handling systems, which are preferably computerized and may be software-driven. Single or multiple capture substrates having the same identifier may be placed together in a collection vessel by the sorting scheme, although in preferred embodiments, multiple capture substrates are collected together to increase the total amount of PBP collected in a sort thereby improving the ability of the assay to characterize bound PBPs that initially may have been present in the sample only in small amounts. In yet other embodiments, sorted capture substrates may be individually transferred or directed to an apparatus for characterizing bound PBPs. In other preferred embodiments, groups of sorted capture substrates may be transferred or directed to an apparatus for characterizing bound PBPs. This latter embodiment is particularly useful when the amount of PBP present on an individual capture substrate provides insufficient material to obtain an adequate signal to noise ratio in the subsequent characterization.

Sorting of the captured PBPs results in separate populations of PBPs in the different collection vessels so that each population (which may include only a single member) represents the different types of PBPs in a given sample that specifically bind to a given capture substrate polynucleotide segment under a given set of capture conditions.

The sorted, bound PBP(s) are then characterized through analytical techniques, which also may include computerized methods. For example, microsequencing, proteolytic cleavage, and/or mass spectrometric methods may be practiced to obtain sequence information about the PBP(s), including the entire sequence of a PBP. Computerized database searching techniques may be incorporated to allow for identification of the PBP, even using partial characterization data such as the mass or mass fragmentation profile of a single peptide. The information obtained by the various characterization techniques may be provided in computer-readable form, and may be compiled in the form of a database.

The invention provides significant advantages in that it allows for the multiplexed performance of multiple assays simultaneously on a single sample.

Multiplexed methods are provided employing at least 2, 3, 4, 5, 10, 15, 20, 25, 50, 100, 200, 300, 400, 500, 1000, 2000, 3000, 5000 or more different capture substrates which may be practiced to simultaneously assay for at least 1, 2, 3, 4, 5, 10, 15, 20, 25, 50, 100, 200, 300, 400, 500, 1000, 2000, 3000, 5000 or more different PBPs in a sample.

Methods amenable to multiplexing, such as those taught herein, allow acquisition of greater amounts of information from a single sample, and thus may reduce the need for sample acquisition, assay time, and/or cost.

The capture substrate comprises at least one surface which is linked to polynucleotide segments, directly or indirectly. The capture substrate also comprises an identifier so that its identity, and optionally, the identity of the polynucleotide segments bound to it, may be determined. In embodiments of the invention in which the identity of the polynucleotide segments is known, and the association between the known polynucleotide segments and their unique identifiers is known, simply determining that a PBP has bound to a particular capture substrate allows the polynucleotide sequence recognized by the PBP to be identified (simultaneously with the detection of PBP binding, or afterwards), without the need for laborious sequencing of the polynucleotide, and therefore may be done with smaller amounts of polynucleotide and PBP, and thus fewer and/or smaller capture substrates.

Any identifier scheme may be practiced; conveniently, the identifier scheme may employ one or more different transponders, or one or more different fluorophores. Because of the large number of identifiable capture substrates that may be is prepared, large numbers of different polynucleotides and polynucleotide-binding proteins may be interrogated simultaneously.

The binding of PBPs to polynucleotides may optionally be detected prior to, in conjunction with, or subsequent to the sorting step prior to characterization of the PBP. In embodiments in which the binding of PBPs is detected prior to sorting, the PBPs are labeled with a detectable label preferably before the contacting step. Only those capture substrates having an associated label need be sorted, since those lacking the label have not bound a PBP. In a particularly preferred embodiment, the PBPs may be labeled with a fluorescent dye, and a binary “pre-sort” may be carried out to separate those capture substrates having bound PBPs from those having no PBP bound using, e.g., a fluorescence-activated cell sorter or FACS. Similarly, if the PBPs are labeled with a fluorescent dye and the capture substrate identifiers comprise other, distinguishable fluorescent dyes, the above-mentioned “pre-sort” may be carried out in conjunction with the sorting step in which those capture substrates having bound PBP are sorted according to the color of their identifier. A PBP may be separated from the capture substrate to which it is bound prior to characterization, or the PBP may be subjected to treatments permitting its characterization while still bound to the capture substrate. Proteins may be separated from the capture substrates by elution, digestion or any other suitable technique. Analytical techniques may then used to provide structural information for the entire captured PBP or portions thereof, for example from peptides obtained by enzymatic digest of the PBPs.

In related embodiments, the invention may be practiced to study alterations of the expression and/or activity of PBPs in response to various stimuli or cell states. The cell populations to be compared may be from a single celled organism, a cultured cell line, a primary cell isolate, a mixed cell culture, a tissue, etc. For example, sequential or simultaneous assays may be carried out on two or more different cell types, or similar cell types subjected to different conditions in an assay of interest, for example to different growth media, drugs, hormones, toxins, pressures, atmospheric compositions, lo temperatures, etc. As one of ordinary skill readily will appreciate, this may be accomplished sequentially by running multiple assays using PBPs prepared from cells exposed to various stimuli, and comparing the data obtained in the different assays. Such comparisons may be practiced to give rise to profiles of PBPs/polynucleotide segment binding pairs associated with one or more stimuli or cell states.

Data also may be obtained from an assay in which samples from each cell type or cell state are obtained and mixed together prior to contacting the capture substrates with the mixed sample. Distinguishable labels optionally may be linked to the proteins from each sample to aid analysis, or unlabeled proteins may be used. The mixed samples may then be tested for the presence of PBPs by detecting binding to the polynucleotide segments on the capture substrates. The relative binding of the PBPs from the different samples may then be directly compared in a competitive assay, in similar fashion to that used to determine relative RNA expression in different test populations using DNA capture substrate array technology.

Similarly, the invention also may be practiced to characterize the binding of known or suspected PBPs to putative binding sites, including characterizing synthetic (e.g., chemically synthesized or recombinant) putative PBPs of unknown specificity, and to characterize the relative affinities of different binding sites for a given PBP. These embodiments of the present invention are particularly useful for developing PBPs or polynucleotide segment PBP binding sites having optimized affinity for use as reagents to control gene expression in, e.g., gene therapy applications, or as reagents for use in displacement assays to develop, e.g., small molecule compounds that affect the interaction between PBPs and their polynucleotide segment binding sites.

Such assays have obvious utility for drug discovery. For example, small molecule compounds identified by such methods that disrupt oncogene expression by interfering with PBP/polynucleotide segment binding driving oncogene expression provide lead compounds for the development of new classes of antineoplastic drugs. The methods described herein also may be practiced to characterize PBPs among a plurality of different proteins from a sample, for example the protein content of a population of cells, and may include assaying the entire protein content of an organism for binding to the entire organism genome.

The methods of the invention also may be practiced to analyze the ability of the PBP(s) in the sample to bind to the polynucleotides on the capture substrates under different sets of capture conditions. Any capture condition that may be varied may be tested for its effect on the ability of the PBP(s) in the sample to bind. Exemplary capture conditions that may be varied include temperature, ionic strength, ionic composition, pH, and osmolarity. See, e.g., Roder K, Schweizer M., “Running-buffer composition influences DNA-protein and protein-protein complexes detected by electrophoretic mobility-shift assay (EMSA),” Biotechnol Appl Biochem 2001 June;33(Pt 3):209-14 and references cited therein (incorporated herein by reference).

The invention may be practiced with PBPs and/or polynucleotides known or unknown to participate in a PBP-polynucleotide interaction. The methods may be used to locate PBPs with binding specificity to a desired polynucleotide. As described above, newly-identified interactions may provide new targets for drug screening as well as providing a screening assay for performing such screening.

The invention may be practiced with naturally occurring PBPs, or may be practiced with non-natural PBPs which have been subject to manipulations. The non-natural PBPs may be partially or entirely of synthetic origin. When applied to PBPs with unknown binding specificities, the methods of the invention may be practiced to characterize their binding specificity.

Before the present invention is described in further detail, it is to be understood that this invention is not limited to the particular methodology, devices, solutions or apparatuses described, as such methods, devices, solutions or apparatuses may, of course, vary. It is also to be understood that the terminology used herein is for the purpose of describing particular embodiments only, and is not intended to limit the scope of the present invention, which is set by the scope of the appended claims.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

The Capture Substrate

The capture substrate is any material that may be sorted, may be linked to a sufficient quantity of a polynucleotide segment so that binding of a PBP may be detected, to and which comprises an identifier uniquely associated with a distinct polynucleotide segment.

The structure of the substrate must be such to allow for its physical movement from the capture reaction into a collection vessel. Preferably, this is accomplished using an automated apparatus.

The capture substrate may comprise any of a wide range of materials. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof.

Capture substrates may be planar crystalline substrates such as silica based substrates (e.g. glass, quartz, or the like), or crystalline substrates used in, e.g. the semiconductor and microprocessor industries, such as silicon, gallium arsenide and the like.

Silica aerogels may also be used as capture substrates, and may be prepared by methods known in the art. Aerogel substrates may be used as free standing substrates or as a surface coating for another substrate material.

The capture substrate may take any form consistent with the criteria identified above and typically is a bead, pin, pellet, disk, particle, strand, precipitate, optionally porous gel, sphere, capillary. A particularly preferred capture substrate is a microtransponder such as are available from PharmaSeq (250×250×100 um) to which may be attached up to 10⁶ polynucleotides per capture substrate, etc. The substrate may be any form that is rigid or semi-rigid. The substrate may contain raised or depressed regions on which a polynucleotide segment is located. The surface of the substrate may be etched using well known techniques to provide for desired surface features, for example trenches, v-grooves, mesa structures, or the like.

Polymeric microspheres or beads are other especially preferred capture substrates. These may be prepared from a variety of different polymers, including those described above. Preferably, the beads are in the size range of approximately 10 nm to 1 mm, and may be manipulated using normal solution techniques when suspended in a solution. The terms “bead,” “sphere,” “microbead” and “microsphere” are used interchangeably herein. Bead based techniques such as those described in PCT application No. PCT/US93/04145 and pin based methods such as those described in U.S. Pat. No. 5,288,514 (the disclosures of which are herein incorporated by reference) also may be useful in practicing the methods of the invention.

Surfaces on the capture substrate may comprise the same material as the capture substrate or may be made from a different material, and may be coupled to the capture substrate by chemical or physical means. Such coupled surfaces may comprise any of a wide variety of materials, for example, polymers, plastics, resins, polysaccharides, silica or silica-based materials, carbon, metals, inorganic glasses, membranes, or any of the above-listed substrate materials. The surface may be optically transparent and may have surface Si—OH functionalities, such as those found on silica surfaces.

The capture substrate and/or its optional surface are chosen to provide appropriate optical characteristics for the synthetic and/or detection methods used. The capture substrate and/or its surface is generally resistant to, or is treated to resist, the conditions to which it is to be exposed in use, and optionally may be treated to remove any resistant material after exposure to such conditions.

The capture substrate and/or its surface may comprise material with low-protein binding characteristics, and/or may be treated to decrease protein binding to decrease undesirable non-specific background binding by components of the sample other than PBPs having specificity for the polynucleotide segment on the substrate. Exemplary materials and techniques to decrease nonspecific protein binding are described by Zyomyx, Inc. in U.S. Pat. No. 6,329,209 and Int'l. Pat. Pubs. Nos. WO 01/72458, WO 01/63241, WO 01/62887, WO 01/51912, WO 00/04390, WO 00/04389, and WO 00/04382.

Identifiers

The capture substrates also comprise identifiers uniquely associated with a distinct polynucleotide segment to allow different capture substrates to be identified and so provide a way to sort and collect capture substrates having the same polynucleotide segments. Any identifier scheme which allows for the identification of the substrate without precluding performance of the remaining steps of the methods of the invention may be used. The identifier usually comprises one or more distinctive physical parameters (e.g., dimensions, shape, density, color, surface molecule, radio signal signature, etc.) such that the different capture substrates may be distinguished and sorted, resulting in like capture substrates being collected together. Optionally, if the sequences of the polynucleotide segments are known and associated with their identifiers, then the identifier provides an instant way to identify the polynucleotide sequence associated with a particular capture substrate.

Exemplary identifier schemes using spectrally encoded microspheres encoded with multiple fluorophores and methods of sorting and decoding them suitable in the methods disclosed herein are described in patents and applications assigned to Luminex Corp. including U.S. Pat. Nos. 5,736,330, 6,057,107, 6,139,800 and 6,268,222 and PCT patent applications Int'l. Pubs. Nos. WO 99/19515, WO 99/37814, WO 01/13119 A1, WO 01/13120 A1, WO 01/14589 A2, and WO 01/63284 A2, the disclosures of which each are herein incorporated by reference.

Exemplary identifier schemes involving encoded microtransponders and nanotransponders that emit distinctive radio signals and methods of sorting and decoding them suitable in the methods disclosed herein are described by PharmaSeq Corp. in patents and applications including U.S. Pat. Nos. 5,641,634, 5,736,332, 5,981,166, 6,001,571, 6,046,003, and 6,051,377, U.S. Pat. App. Pub. No. US 2001/0044109 A1 and PCT patent applications Int'l. Pubs. Nos. WO 97/19958, WO 97/20073, and WO 97/20074, the disclosures of which each are herein incorporated by reference. These microtransponders comprise an integrated circuit comprising photocells, memory, clock and antenna. The codes may be read by activating the transponder's memory using a laser and using a high-speed flow fluorometer modified to detect radio frequency from the antenna produced from the transponder.

The Capture Substrate Polynucleotide Segments

A plurality of polynucleotide segments of different sequences are attached to capture substrates that also comprise identifiers that are uniquely associated and so identify capture substrates having polynucleotide segments of the same sequence. Similarly, capture substrates having different identifiers each will have polynucleotide segments of differing sequence (i.e., distinct polynucleotide segments) associated with them. The polynucleotide segments may be single-stranded, double-stranded, or higher order, and may be linear, branched, multimeric or circular, and may contain nonnatural bases. Typically, the polynucleotide segments are linear single-stranded (“ss”) or double-stranded (“ds”) DNA or RNA, and most preferably comprise double stranded DNA.

Preferred size ranges for dsDNA polynucleotide segments include from about 4 to about 5000 nucleotides or from about 4 to about 1000 nucleotides, or from about 6 to about 500 nucleotides or from about 10 to about 450 nucleotides, or from 10 to about 300 nucleotides, or from about 10 to about 250 nucleotides, or from about 10 to about 200 nucleotides, or from about 10 to about 150 nucleotides, or from about 10 to about 100 nucleotides, or from about 4 to about 100 nucleotides, or from about 4 to about 50 nucleotides, or from about 6 to about 25 nucleotides in length. In one preferred embodiment, all capture substrates used in an assay are conjugated with polynucleotide segments having the same length. In another embodiment, different capture substrates are conjugated with polynucleotide segments having different lengths.

In one embodiment of the invention, localization of the sequence contained within the polynucleotide segment recognized by the PBP may be effected using techniques known to those of skill in the art. These include footprinting studies, in which regions of DNA bound to protein are protected from protease digestion, restriction site masking, and other well-known techniques such as are disclosed in U.S. Pat. No. 6,100,035 (which is herein incorporated by reference) and fine-structure mapping in which a longer polynucleotide segment is broken into shorter segments which then are used to construct additional capture substrates. For example, if a first assay reveals that a given PBP binds to a polynucleotide segment of 100 base pairs length, that segment may be broken into four 25 base-pair segments that are attached to four different capture substrates that then are assayed for their ability to bind the given PBP.

Exemplary single-stranded polynucleotide segments include total RNA, poly(A)+RNA, mRNA, rRNA, tRNA, hnRNA, and ssRNA and ssDNA viral genomes, although these polynucleotides may contain internally complementary sequences and significant secondary structure. Exemplary double-stranded polynucleotide segments include genomic DNA, mitochondrial DNA, chloroplast DNA, double-stranded oligonucleotides, organism genomes, organism genes, upstream regions of known or suspected genes, dsRNA or dsDNA viral genomes, plasmids, phage, and viroids. The polynucleotide segment may be prepared synthetically or purified from a biological source. Where the polynucleotide segment is purified from a biological source, nuclei or any other subcellular fraction may be separated prior to obtaining an extract comprising the PBP(s).

The polynucleotide segments optionally may be purified to remove and/or diminish one or more undesired components and/or to concentrate the polynucleotide segments prior to amplification. Conversely, where the polynucleotide segment is too concentrated for a particular assay, the polynucleotide segments may first be diluted.

The polynucleotide segments may be synthesized directly on the encoded is substrate, or may be synthesized separately from the substrate and then coupled to it. Suitable methods are disclosed in U.S. Pat. No. 6,100,035, which is herein incorporated by reference. Direct synthesis on the substrate may be accomplished by incorporating a monomer or other functional group that is coupled to a subunit of the polynucleotide segment into a polymer or other material that makes up or is deposited on or coupled to the substrate, and then synthesizing the remainder of the polynucleotide segment to incorporate that subunit. Or the substrate or its coating may include or be derivatized to include a functional group which may be coupled to a subunit of the polynucleotide segments for synthesis, or may be coupled directly to the complete polynucleotide segment. See, e.g., Beaucage S L, “Strategies in the preparation of DNA oligonucleotide arrays for diagnostic applications,” Curr Med Chem. 2001 August;8(10):1213-44, which is herein incorporated by reference. Polynucleotide segments may be fabricated on or attached to the capture substrate by any suitable method, for example the methods described in U.S. Pat. No. 5,143,854, PCT Publ. No. WO 92/10092, Fodor et al., Science, 251: 767-777 (1991), and PCT Publ. No. WO 90/15070). Mechanical synthesis strategies are described in, e.g., PCT Publication No. WO 93/09668 and U.S. Pat. No. 5,384,261, the disclosures of which are herein incorporated by reference.

Additional flow channel or spotting methods applicable to attachment of polynucleotide segments to the substrate are described in U.S. patent application Ser. No. 07/980,523, filed Nov. 20, 1992, and U.S. Pat. No. 5,384,261, the disclosures of which are herein incorporated by reference. Reagents are delivered to the substrate by either (1) flowing within a channel defined on predefined regions or (2) “spotting” on predefined regions. A protective coating such as a hydrophilic or hydrophobic coating (depending upon the nature of the solvent) may be practiced over portions of the substrate to be protected, sometimes in combination with materials that facilitate wetting by the reactant solution in other regions. In this manner, the flowing solutions are further prevented from passing outside of their designated flow paths.

Typical dispensers useful for reagent delivery for the above-described methods include a micropipette optionally robotically controlled, an ink-jet printer, a series of tubes, a manifold, an array of pipettes, or the like so that various reagents may be delivered to the reaction regions sequentially or simultaneously.

Linking molecules may be used to link the polynucleotide segment to the substrate. Examples of suitable spacers or linkers are polyethyleneglycols, dicarboxylic acids, polyamines and alkylenes. The spacers or linkers are optionally substituted with functional groups, for example hydrophilic groups such as amines, carboxylic acids and alcohols or lower alkoxy group such as methoxy and ethoxy groups. The spacers may have an active site on or near a distal end. The active sites are optionally protected initially by protecting groups. Among a wide variety of protecting groups which are useful are FMOC, BOC, t-butyl esters, t-butyl ethers, and the like. Various exemplary protecting groups are described in, for example, Atherton et al., Solid Phase Peptide Synthesis, IRL Press (1989).

A plurality of polynucleotide segments of the same sequence are attached to capture substrates having the same identifier. A PBP having degenerate binding specificity may bind to more than one polynucleotide segment, with the same or different affinities. The methods disclosed herein may be practiced to characterize the specificity and affinity of different PBPs for different polynucleotide segments. By “different” polynucleotide segments is meant polynucleotides differing in polynucleotide sequence in at least one position.

The Sample

The sample comprising or suspected of comprising PBP(s) may be from any source. The source of the sample may be a biological source from which the sample is purified and/or isolated, including natural and artificial biological sources including cultured cells. The sample may also comprise a PBP prepared through synthetic means, in whole or in part. The sample may comprise a protein produced using in vitro transcription/translation systems, for example from the rabbit reticulocyte lysate or pSP64 systems.

Typically, the sample is obtained as and/or dispersed in a predominantly aqueous medium. Nonlimiting examples of natural biological sources from which the sample may be obtained include blood, urine, semen, milk, sputum, mucus, a buccal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs.

Nonlimiting examples of artificial biological sources from which the sample may be obtained include cultured cells, either genetically manipulated or not. The cultured cells may be genetically altered to express a PBP, which then may be partially or completely isolated and/or purified from the cells. The cultured cells may be a cell population expressing multiple proteins from an expression library, including up to the entire expressed proteome of an organism. The cultured cells may be a cell line or a primary isolate, and may be a mixed cell population comprising more than one type of cell. The cells may be normal cells, mutated cells, genetically manipulated cells, tumor cells, etc.

The sample may be a positive control sample which is known to contain a PBP. A negative control sample may also be used which, although not expected to contain the PBP, is suspected of containing it, and is tested in order to confirm the lack of contamination of the reagents used in a given assay, as well as to determine whether a given set of assay conditions produces false positives (a positive signal even in the absence of PBPs in the sample).

Where the sample is obtained from a natural source, the natural source may be of any origin, including prokaryotes, eukaryotes, or archeons. The cell(s) may be living or dead. If obtained from a multicellular organism, the sample may be from any cell type, tissue, organ, developmental stage, etc. The natural source may be of mammalian, amphibian, reptilian, plant, yeast, bacterial, spirochete, or protozoan origin. Exemplary mammalian sources include humans, mice, rats, cows, pigs, sheep, hamsters, chickens, quail, and dogs. Exemplary cell types include those that may be obtained from the American Type Culture Collection in Manassas, Va. or through its distributors.

Cells in the sample may be lysed or otherwise permeabilized to release the PBPs within the cells. One-step permeabilization buffers may be practiced to lyse cells which allow further steps to be performed directly after lysis. Subcellular fractions may be used to prepare the sample, for example from the nucleus, mitochondria, cytoplasm, chromatin, nucleolus, or any other subcellular organelle or fraction. The sample may be diluted, dissolved, suspended, extracted or otherwise treated to solubilize and/or purify any PBPs present or to render them accessible to reagents which may be used in the practice of the methods disclosed.

The Polynucleotide-Binding Protein(s)

Any protein that may bind to a polynucleotide segment on a capture substrate in an identifiable manner may be employed in the methods disclosed. Non-limiting examples of PBPs include DNA-binding proteins including transcription factors, is splicing factors, poly(A) binding proteins, chromatin components, viral proteins, proteins that detect viral infection, replication factors, and proteins involved in mitotic and/or meiotic cell division. Exemplary DNA-binding proteins include zinc-finger proteins, homeodomain proteins, winged-helix (forkhead) proteins, leucine-zipper proteins, helix-loop-helix proteins, helix-turn-helix proteins, and histone-like proteins.

The PBPs may be isolated from a cell source, or may be produced in vitro, for example through in vitro transcription/translation methods or through completely synthetic methods. The PBPs may be naturally occurring proteins, mutants of naturally occurring proteins, randomly produced proteins produced, for example, by molecular evolution methods, and may include prospective polynucleotide-binding proteins created based on sequences of known PBPs and having previously uncharacterized binding activities. Methods disclosed herein may be practiced to characterize the binding specificities of such proteins.

Capture Conditions

Capture conditions are chosen for incubation of the sample with the capture substrates; factors which may be varied include pH, buffer, ionic strength, salts, cofactors such as nucleotides and metal ions, chelators, optional cosolvents, time, small molecules, and temperature. The results of the assay are determined under a given set of conditions. The conditions may be optimized for a particular interaction of interest. The conditions may also be varied to provide a profile of the binding interaction of one or more PBPs. Aliquots of the sample may be subjected to an array of capture conditions varying in one or more factors to produce binding profiles of the different PBPs contained within the sample. Reagents useful for lowering nonspecific protein binding may be included in the assay, for example blocking solutions including proteins (e.g., BSA), detergents, nonspecific polynucleotides (e.g., repeat sequences, Cotl DNA), etc. The capture substrates may be subjected to one or more washing steps prior to final characterization of the bound PBPs. In preferred embodiments, the assay is multiplexed, i.e., multiple distinct assays are run simultaneously, to allow analysis of multiple PBPs in the sample. In the simplest case, multiplexing is accomplished by incubating a sample suspected to contain one or more PBP with a mixture of capture substrates.

Sorting the Capture Substrates

Contacting the capture substrates with a PBP-containing sample under conditions in which at least one PBP binds a polynucleotide segment results in the formation of a bound capture substrate. The capture substrates then are sorted according to the capture substrate identifiers that are uniquely associated with a distinct polynucleotide segment. Capture substrates having the same identifier are collected together. In preferred embodiments, the sorting is done using an automated method. Any automated sorting method capable of sorting the capture substrates and allowing characterization of bound PBPs may be practiced, for example flow sorting techniques, molecular tweezers (reviewed in Greulich K O, Pilarczyk G., “Laser tweezers and optical microsurgery in cellular and molecular biology. Working principles and selected applications,” Cell Mol Biol (Noisy-le-grand) 1998 July;44(5):701-10, incorporated herein by reference), mechanical systems, electrophoretic separators, magnetic separators, chromatographic separation systems including liquid chromatography, high performance liquid chromatography (“HPLC”), etc.

The sorting system utilizes the identifiers comprising the capture substrates to uniquely identify capture substrates comprising the same polynucleotide segment. In one aspect, a pneumatic liquid handling system may be practiced to sort and collect the capture substrates. Preferably, the sorting system is computer operated, and preferably allows for very high degrees of multiplex sorting. A computerized sorting system may utilize software to control the operation of the automated sorting apparatus.

The particular structure of the collection vessels are not critical; the collection vessels are used to retain sorted captured substrates, and steps to characterize PBPs bound thereto may be performed in the vessel. Individual collection vessels may be completely separate from one another (e.g., individual test tubes) or may be connected into higher order structures, such as multiwell dishes or plates. The vessels typically are not in fluid contact with one another, but may take the form of wells or other structures in a single plate in which the sorted capture substrates may be deposited, for example using molecular tweezer sorting methods. A given collection vessel ideally contains only one type of capture substrate, and may contain a single capture substrate, 2, 3, 4, 5, 10, 20, 50, 100, 200, 300, 400, 500, 1000, 2000, 5000 or more capture substrates, depending on the scale of the experiment, the size of the capture substrates and the capacity of the vessel.

The binding of PBPs to polynucleotides may optionally be detected prior to, concurrent with, or subsequent to sorting of the capture substrates from the sample. Detection of PBP binding may be performed by any suitable technique. The technique may be one that merely detects the binding of a PBP to a capture substrate, one which detects the absolute and/or relative amount of binding, or one which characterizes the PBP(s) which are bound to a given capture substrate. Exemplary methods which may be practiced to detect PBP binding include optical, spectroscopic, electrical, piezoelectrical, magnetic, Raman scattering, surface plasmon resonance, radiographic, colorimetric, and calorimetric detection methods. Of course, capture substrates without bound PBPs also may be sorted during the practice of the invention, but these do not serve as sources for PBPs for further characterization.

Sorting produces a population of bound capture substrates in a given collection vessel. The PBPs captured on these capture substrates represent a profile of PBPs in the sample which bind under the set of capture conditions tested. For example, the profile may represent the set of PBPs obtained from a normal cell, or from a malignant cell, or from a cell exposed to a test compound such as a drug. The populations of the different collection vessels measured against the array of different polynucleotide segments used in a given experiment may provide PBP binding profiles for the entire protein content of a tissue or organism. The binding profiles may be measured against the entire genome content of an organism, or against the entire collection of known upstream regulatory regions, for example. The invention described herein allows characterization of the capture profile of PBPs from any of a diverse range of samples obtained under any desired set of conditions against any desired array of polynucleotide segments.

Characterization of PBPs

PBPs captured on capture substrate and sorted in the collection vessels are then characterized, preferably in a manner that yields some type of structural information. Structural information may be obtained from the entire PBP or any fragment thereof. In one aspect, the PBPs are fragmented into peptides, either while bound to the capture substrates or after dissociation from the capture substrates. Any technique which fragments the PBPs while permitting structural information to be obtained may be used; a number of techniques are known in the art. Exemplary methods of fragmentation include protease digestion (e.g. using trypsin, chymotrypsin, staphylococcus V8 protease, etc.), chemical techniques (e.g. hydroxyurea, acidic conditions, etc.), and mass spectrometric techniques. The PBPs or peptides derived therefrom may be dissociated from the capture substrates using any method which does not impede the acquisition of structural information; suitable techniques are known in the art. Exemplary methods and reagents which may be practiced to dissociate the PBPs or fragments thereof from the capture substrates include heating, addition of chaotropic agents, chelating agents, solvents, or detergents, altering the ionic strength or composition, enzymatic digestion, electrophoretic methods, and combinations thereof.

Characterization preferably includes methods by which structural information may be obtained from the peptides directly; the peptides initially generated may also be subject to further fragmentation to allow acquisition of additional structural information about the PBPs from these additional fragments.

Any method that characterizes the bound PBPs by providing structural information for PBPs that have bound to the capture substrates may be practiced in conjunction with this invention. The PBPs or PBP fragments may be characterized by electrophoretic techniques such as SDS-PAGE and capillary electrophoresis, by microbore liquid chromatography, by chromatographic methods, immunological techniques (e.g., western blots, ELISAs, competition assays, sandwich assays, etc.), by mass spectrometry (MS), by HPLC optionally with electrochemical detection, by circular dichroism (CD), by X-ray or electron diffraction methods, by spectroscopic methods including, e.g., infra-red spectroscopy, Fourier transformed infra-red spectroscopy (“FTIR”), Raman spectroscopy, or nuclear magnetic resonance (NMR) spectroscopy, by traditional protein detection methods (for example the Lowry assay (Lowry O H, Rosenbrough N J, Fass A L, Randall R J., “Protein measurement with the Folin phenol reagent,” J Biol Chem 193:265-275 (1951) (incorporated herein by reference), and the Bradford assay (Bradford M M, “A rapid and sensitive method for the quantitation of microgram quantities of protein utilizing the principle of protein-dye binding,” Anal Biochem, 72: 248-254 (1976) (incorporated herein by reference)), by protein sequence techniques (Edman degradation (Edman P, Acta Chem Scand 4: 253 (1950) or Matsudaira, P., “Sequence from picomole quantities of proteins electroblotted onto polyvinylidene difluoride membranes,” J. Biol. Chem. 262:10035-10038 (1987), (both of which are herein incorporated by reference) etc.), by detection of the label coupled to or incorporated in the PBP(s), and/or by spectrophotometry.

Preferred characterization methods provide large amounts of structural information that may include the entire amino acid sequence of the PBP, including matrix-assisted laser desorption ionization-time of flight mass spectrometry (MALDI-TOF) preferably with peptide mass matching, and electrospray mass spectrometry. Mass spectrometry/mass spectrometry (MS/MS) may be practiced, wherein peptides are first separated by MS, ions are selected and further fragmented and the resulting fragments are subjected to an additional round of MS. The patterns of fragments produced provides structural information about the PBP; the actual patterns may be compared to predicted fragmentation patterns for various peptides, preferably using a computer, to provide sequence information about the PBPs.

Direct sequencing methods may be practiced. Methods are known in the art for obtaining microsequence data, for example Fourier Transform Ion Cyclotron Resonance (FTICR), which may generate sequence data from attamole amounts of protein. In combination, such techniques allow sequence data to be obtained from a single sorted captured PBP, thereby allowing for very high levels of multiplexing using minimal sample volumes.

Doping techniques also may be used as internal controls or to confirm the identity of a given peptide where absolute sequence information cannot be obtained. Isotopic doping methods may provide absolute quantitation of the amount of protein bound. Alternatively, isotopic labeling may be practiced for comparative techniques.

The invention may be performed in an automated fashion to allow high-throughput analysis of multiple PBPs against a liquid array of polynucleotide segments. Robots may be used to perform any or all of the steps described herein. Computers may be practiced to implement the methods described herein, and the structural information obtained from the methods may be compiled in a computer-readable form, for example in the form of a database. Structural information obtained from a fragment and/or the complete PBP may be practiced in combination with search algorithms that preferably are computer-implemented to search databases and used to identify the PBP or a homolog thereof; for example, a BLAST search may be performed on a database using sequence information obtained from a peptide obtained from the captured PBP, for example by mass spectrometry, to identify the PBP in a database, and/or to identify homolog(s) of the PBP contained in the database, or, e.g., to compare proteolytic fragment profiles experimentally obtained with those predicted for proteins of known primary structure.

Labels

A label may optionally be attached to the PBP(s) in a sample to allow the capture of the PBP(s) to be more easily detected. Where the PBPs from multiple samples are tested in competitive assays, the labels attached to the PBPs in the different samples are distinguishable. The label is attached, directly or indirectly, to molecules in the sample, including PBPs present therein. Many labels are commercially available in activated forms which may readily be used for such conjugation (for example through amine acylation), or labels may be attached through known or determinable conjugation schemes many of which are well-characterized in the art.

Labels useful in the invention described herein include any substance which may be detected in association with the substrate when the PBP to which the label is attached is bound to the polynucleotide segment. Any effective detection method may be practiced, including optical, spectroscopic, electrical, piezoelectrical, magnetic, Raman scattering, surface plasmon resonance, radiographic, colorimetric, calorimetric, etc.

The label typically comprises an agent selected from a chromophore, a lumiphore, a fluorophore, a chromogen, a hapten, an antigen, a radioactive isotope, a magnetic particle, a metal nanoparticle such as a gold or silver nanoparticle, an enzyme, an antibody or binding portion or equivalent thereof, an aptamer, one member of a binding pair, and combinations thereof.

A fluorophore may be any substance that absorbs light of one wavelength and emits light of a different wavelength. Typical fluorophores include fluorescent dyes, semiconductor nanocrystals, lanthanide chelates, and a green fluorescent protein.

Exemplary fluorescent dyes include, e.g., fluorescein, 6-FAM, rhodamine, Texas Red, Cy2®, Cy3®, Cy3.5®, Cy5®, and Cy5.5® and others, many of which may be obtained from Molecular Probes, Inc. (Eugene, Oreg.) or from Amersham Biosciences (Piscataway, N.J.) or from other suppliers commonly known to those of skill in the art.

A wide variety of fluorescent semiconductor nanocrystals are known in the art; methods of producing and utilizing semiconductor nanocrystals are described in: PCT Publ. No. WO 99/26299 published May 27, 1999, inventors Bawendi et al.; U.S. Pat. No. 5,990,479 issued Nov. 23, 1999 to Weiss et al.; U.S. Pat. No. 6,274,323 B1 issued Aug. 14, 2001 to Bruchez et al., and Bruchez et al., Science 281:2013, 1998, the disclosure of which are each herein incorporated by reference. Exemplary lanthanide chelates include europium chelates, terbium chelates and samarium chelates.

The term “green fluorescent protein” refers to both native Aequorea green fluorescent protein and mutated versions that have been identified as exhibiting altered fluorescence characteristics, including altered excitation and emission maxima, as well as excitation and emission spectra of different shapes (Delagrave, S. et al. (1995) Bio/Technology 13:151-154; Heim, R. et al. (1994) Proc. Natl. Acad. Sci. USA 91:12501-12504; Heim, R. et al. (1995) Nature 373:663-664 (each of which is herein incorporated by reference)). Delgrave et al. isolated mutants of cloned Aequorea victoria GFP that had red-shifted excitation spectra. Bio/Technology 13:151-154 (1995). Heim, R. et al. reported a mutant (Tyr66 to His) having a blue fluorescence (Proc. Natl. Acad. Sci. (1994) USA 91:12501-12504).

Exemplary enzymes include alkaline phosphatase, horseradish peroxidase, β-galactosidase, glucose oxidase, galactose oxidase, neuraminidase, a bacterial luciferase, an insect luciferase and sea pansy luciferase (Renilla koellikeri), which may create a detectable signal in the presence of suitable substrates and assay conditions, known in the art.

Exemplary haptens and/or members of a binding pair include avidin, streptavidin, digoxigenin, biotin, and those described above.

Kits

Kits comprising reagents useful for performing the methods of the invention are also provided. In one embodiment, a kit comprises a mixture of capture substrates wherein said substrates comprise an identifier and a polynucleotide segment, and wherein each of said identifiers is uniquely associated with a distinct polynucleotide segment. The substrates may be identifiable beads comprising spectral identifiers based on fluorophores, or may be microtransponders which may be activated by light to transmit their stored code from an antenna. A sample may be assayed for polynucleotide binding protein(s) using the components of the kit as disclosed above. Pluralities of different identifiable sortable capture substrates attached to corresponding different polynucleotide segments may be included in the kit to allow for assaying of pluralities of PBPs in the sample.

The kit may comprise a reagent for incorporating a label into the polynucleotide-binding protein. The components of the kit are retained by a housing. Instructions for using the kit to perform a method of the invention are provided with the housing, and may be located inside the housing or outside the housing, and may be printed on the interior or exterior of any surface forming the housing which renders the instructions legible.

### EXAMPLES

The following examples are set forth so as to provide those of ordinary skill in the art with a complete description of how to make and use the present invention, and are not intended to limit the scope of what is regarded as the invention. Efforts have been made to ensure accuracy with respect to numbers used (e.g., amounts, temperature, etc.) but some experimental error and deviation should be accounted for. Unless otherwise indicated, parts are parts by weight, temperature is degree centigrade and pressure is at or near atmospheric, and all materials are commercially available.

### Example 1

The following experiment is performed to isolate and identify multiple DNA-binding proteins:
1. A plurality of double-stranded DNA sequences of interest are synthesized.
2. The DNA strands are attached to the surfaces of identifiable capture substrates to form a set of identifiable capture substrates. Each distinct DNA sequence is attached to capture substrates having the same identifier so that an identifier is uniquely associated with a distinct DNA sequence.
3. A cell extract is prepared that is suspected to contain DNA binding proteins.
4. Components of the cell extract are labeled with a fluorescent probe.
5. The labeled cell extract is mixed with the set of identifiable capture substrates.
6. Aliquots of the cell extract mixed with the encoded capture substrates are subjected to different capture conditions to determine their effect on DNA binding. Capture conditions which are varied include salt concentration, pH, temperature, time and the presence of inhibitors and specific cofactors in the mix.
7. After incubation of the mixture for a suitable time to allow binding, the encoded capture substrates are passed through the excitation beam and fluorescence of any bound protein is measured. This is done in a liquid stream though which the capture substrates flow.
8. If a detectable amount of protein is bound to the capture substrate, the capture substrate is sorted into a specific collection tube or well. Capture substrates having the same identifier are collected together to increase the amount of protein for further analysis.
9. After sorting, proteins attached to the capture substrates are subject to extraction for electrophoretic or chromatographic separation or are directly fragmented, for example with a protease such as trypsin, for the production of peptides. Aliquots of the sorted capture substrates may be separately subjected to different techniques.
10. The peptides are extracted and subjected to mass spectrometry.
11. Two types of mass spectrometry are used, MALDI-TOF with peptide mass matching for identification and MS/MS peptide fragmentation followed by subsequent database identification.
12. The identified PBP is associated in a database with the polynucleotide sequence to which it bound on the capture substrate, and optionally with the binding conditions, the cell type, cell-state, or cell treatment preceding sample preparation.

Although the invention has been described in some detail with reference to the preferred embodiments, those of skill in the art will realize, in light of the teachings herein, that certain changes and modifications may be made without departing from the spirit and scope of the invention. Accordingly, the invention is limited only by the claims.

1. A method for characterizing polynucleotide binding proteins, comprising:
providing a mixture of capture substrates, said substrates comprising a polynucleotide segment and an identifier uniquely associated with a distinct polynucleotide segment;
contacting said capture substrates with a sample comprising at least one polynucleotide binding protein under conditions in which said at least one polynucleotide binding protein binds to said polynucleotide segment;
sorting said capture substrates to collect substrates having the same identifier; and
characterizing at least one polynucleotide binding protein bound to at least one collection of sorted capture substrates.

2. The method of claim 1, wherein said substrate is selected from the group consisting of a bead, a chip, and a pin.

3. The method of claim 1, wherein said identifier is selected from the group consisting of a radio signal, a chromophore, a lumiphore, a fluorophore, a chromogen, an antigen, a radioisotope, a magnetic particle, a metal nanoparticle, an enzyme, an antibody, an antibody fragment, an aptamer, a hapten, an antigen, a size, a density, a mass, a shape, and a radioisotope.

4. The method of claim 3, wherein said identifier is a radio signal.

5. The method of claim 1, wherein said polynucleotide segment is selected from the group consisting of DNA and RNA.

6. The method of claim 5, wherein said DNA is double stranded.

7. The method of claim 5, wherein said DNA is single stranded.

8. The method of claim 1, wherein the sequence of said polynucleotide segment is known.

9. The method of claim 1, wherein said polynucleotide binding proteins are detectably labeled prior to said contacting step.

10. The method of claim 1, wherein said detectable label is selected from the group consisting of a chromophore, a lumiphore, a fluorophore, a chromogen, a hapten, an antigen, a radioisotope, a magnetic particle, a metal nanoparticle, an enzyme, an antibody, an antibody fragment, an aptamer, a hapten, an antigen, and a radioisotope.

11. The method of claim 1, wherein said sorting is carried out manually.

12. The method of claim 1, wherein said sorting is automated.

13. The method of claim 12, wherein said automated sorting comprises the use of a FACS, a transponder reader, a pneumatic liquid handling system, or an electroosmotic liquid handling system.

14. The method of claim 13, wherein said automated sorting comprises the use of a transponder reader.

15. The method of claim 1, wherein said characterizing step comprises obtaining sequence data from said at least one bound polynucleotide binding protein.

16. The method of claim 15, wherein said sequence data is partial.

17. The method of claim 1, wherein said characterizing step comprises obtaining a mass of said at least one bound polynucleotide binding protein.

18. The method of claim 1, wherein said characterizing step comprises digesting said at least one bound polynucleotide binding protein with a protease and characterizing a resulting proteolytic fragment of said polynucleotide binding protein.

19. The method of any one of claims 15, 16, 17, and 18, further comprising comparing the results obtained in said characterizing step to data contained within a database.

20. The method of claim 1, further comprising cleaving said polynucleotide segment at a known location and determining whether said at least one polynucleotide binding protein remains bound to said capture substrate.

21. The method of claim 1, wherein said sorted substrates are collected in a plurality of collection vessels.

22. A kit for assaying a sample for a polynucleotide binding protein comprising:
a mixture of capture substrates wherein said substrates comprise an identifier and a polynucleotide segment, and wherein each of said identifiers is uniquely associated with a distinct polynucleotide segment;
a housing for retaining said mixture of capture substrates; and
instructions provided with said housing that describe how to perform the method of claim 1.

23. The kit of claim 20, further comprising a reagent comprising a label suitable for conjugation to proteins in said sample, and instructions that describe how to perform the method of claim 9.
